(19) United States
(12) Patent Application Publication
(10) Pub. No.: US 2026/0215811 A1
(43) Pub. Date: Jul. 30, 2026

(54) MEDICAL SYSTEM AND APPROACH METHOD

(71) Applicant: TERUMO KABUSHIKI KAISHA, Tokyo (JP)

(72) Inventors: Katsuhiko SHIMIZU, Fujinomiya-shi (JP); Hiroyuki ISHIHARA, Setagaya-ku (JP)

(73) Assignee: TERUMO KABUSHIKI KAISHA, Tokyo (JP)

(21) Appl. No.: 19/629,757

(22) Filed: Mar. 26, 2026

Related U.S. Application Data
(63) Continuation of application No. PCT/JP2024/032488, filed on Sep. 11, 2024.

(30) Foreign Application Priority Data
Sep. 29, 2023 (JP) 2023-169567

(51) Int. Cl. A61B 17/34; A61B 17/00; A61B 90/00

(52) U.S. Cl. CPC A61B 17/3417; A61B 17/00234; A61B 90/39; A61B 2017/00243; A61B 2017/003; A61B 2017/00477; A61B 2090/3925; A61B 2090/3966

(57) ABSTRACT

A medical system including: an elongated guiding catheter having flexibility; an elongated puncture tool that is insertable into the guiding catheter, has a puncture needle at an distal portion, and includes a lumen extending from a proximal portion to the distal portion and a distal opening communicating with the lumen at the distal portion; a biasing tool that includes a movement portion movable for applying a biasing force toward a distal direction to a material passing through the lumen and a biasing portion that biases the movement portion; and a releasable stopper that is capable of fixing the movement portion at a predetermined position.

### CROSS-REFERENCES TO RELATED APPLICATIONS

This application is a continuation of International Application No. PCT/JP2024/032488 filed on September 11, 2024, which claims priority to Japanese Application No. 2023-169567 filed on September 29, 2023, the entire content of both of which is incorporated herein by reference.

### TECHNOLOGICAL FIELD

The present disclosure relates to a medical system and an approach method capable of approaching a pericardial space via a blood vessel.

### BACKGROUND DISCUSSION

In recent years, treatment such as performing arrhythmia ablation through a pericardial space, which is located between two layers of the pericardium constituting the pericardial sac covering myocardial tissue, draining a body fluid from the pericardial space, or supplying agents thereto is performed. For such treatment, for example, Japanese Patent Application Publication No. 2017-506560 A discloses a method and a device for approaching the pericardial space from the body surface of a patient.

Since parts of two layers of the pericardium are in close contact with each other, the pericardial space is extremely narrow. For this reason, in order to approach the pericardial space from the body surface, it requires measures to widen a gap between the two layers of the pericardium, and the procedure is complicated. There also is a possibility of puncturing an unintended position of the heart with a needle, and the safety is relatively low.

### SUMMARY

A medical system and an approach method are disclosed, which are capable of safely and easily approaching a pericardial space.

(1) According to the present disclosure, there is provided a medical system including: an elongated catheter having flexibility; an elongated puncture tool that is insertable into the catheter, has a puncture needle at an distal portion of the puncture tool, and includes a lumen extending from a proximal portion of the puncture tool to the distal portion of the puncture tool and a distal opening communicating with the lumen at the distal portion of the puncture tool; a biasing tool that includes a movement portion movable for applying a biasing force toward a distal direction to a material passing through the lumen and a biasing portion that biases the movement portion; and a releasable stopper that is capable of fixing the movement portion at a predetermined position.

In the medical system according to (1), when the stopper is released, the movement portion can apply the biasing force to the material passing through the lumen of the puncture tool and push the material from the distal end of the puncture needle to the outside. Therefore, since the medical system can cause the material passing through the lumen of the puncture tool to reach the pericardial space by puncturing the inner wall of the heart or the inner wall of the blood vessel with the puncture tool from the inside of the heart chamber or the inside of the blood vessel, the puncture tool can be safely and easily caused to approach the pericardial space while observing the material reaching the pericardial space through the lumen.

(2) The medical system according to (1) may further include a visualization means having ultrasonic echogenicity or X-ray radiopacity as the material passing through the lumen. Thus, in the medical system, the visualization means that reaches the pericardial space through the lumen can be observed by using an imaging device using ultrasonic waves or X-rays.

(3) In the medical system according to (2), the visualization means may be a wire. Thus, in the medical system, the wire reaching the pericardial space through the lumen can be observed using the imaging device.

(4) In the medical system according to (2), the visualization means may be a liquid containing a plurality of microbubbles. Thus, in the medical system, the microbubbles reaching the pericardial space through the lumen can be observed using the imaging device.

(5) In the medical system according to any one of (1) to (4), the biasing tool may be detachable from the puncture tool. Thus, since the biasing tool can be attached to the puncture tool when necessary and removed from the puncture tool when unnecessary, the workability of the puncture tool can be improved.

(6) In the medical system according to any one of (1) to (5), the biasing tool may include a wire fixing portion capable of fixing the wire to the movement portion. Thus, the medical system can easily fix the wire to the movement portion and move the wire together with the movement portion.

(7) According to an embodiment, a medical system comprising: an elongated catheter; an elongated puncture tool configured to be inserted into a lumen of the catheter, the puncture tool including an elongated shaft having a puncture needle at a distal end of the elongated shaft, and wherein the elongated shaft includes a lumen extending from a proximal end of the elongated shaft to the distal end of the elongated shaft and a distal opening communicating with the lumen at the distal end of the elongated shaft; and a biasing tool that includes a movement portion configured to be movable for applying a biasing force toward a distal direction to a material passing through the lumen and a biasing portion configured to bias the movement portion.

(8) According to the present disclosure, there is provided an approach method for approaching a pericardial space from an inside of a heart chamber or an inside of a blood vessel, the method including: preparing an elongated catheter having flexibility and an elongated puncture tool which is insertable into the catheter, has a puncture needle at a distal end, and includes a lumen extending from a proximal portion to a distal portion and a distal opening communicating with the lumen at the distal portion; inserting the catheter into the blood vessel and delivering the catheter to a puncture position in the heart chamber or the blood vessel from which the puncture tool inserted into the catheter is capable of performing a puncture toward the pericardial space; at the puncture position, pressing an inner wall of a heart or an inner wall of the blood vessel toward the pericardial space at the distal portion disposed at the distal end of the catheter to make a tenting state; making a puncture preparation state by imaging the pressed inner wall of the heart or the pressed inner wall of the blood vessel using an imaging device, biasing a visualization means, which has ultrasonic echogenicity or X-ray radiopacity and is inserted into the lumen, in a direction to push the visualization means out of the lumen from the distal opening of the puncture tool, and restricting movement of the visualization means to an outside from the distal opening; and in the tenting state and the puncture preparation state, puncturing the inner wall of the heart or the inner wall of the blood vessel with the puncture needle through the lumen of the catheter to release the restriction on the movement of the visualization means, and inserting the biased visualization means into the pericardial space through the distal opening of the puncture tool while imaging by the imaging device.

Thus, in the present approach method, by puncturing the inner wall of the heart or the inner wall of the blood vessel from the inside of the heart chamber and the inside of the blood vessel with the puncture tool, the visualization means passing through the lumen of the puncture tool can reach the pericardial space. Therefore, in the present approach method, the puncture tool and the visualization means can approach the pericardial space safely and easily by observing the visualization means using the imaging device.

### BRIEF DESCRIPTION OF THE DRAWINGS

FIG. 1 is a plan view illustrating a medical system according to a first embodiment.

FIG. 2 is a cross-sectional view illustrating a medical system according to the first embodiment.

FIG. 3 is a schematic view showing a cross-section view of a living body and a plan view of a medical system and an imaging device.

FIG. 4 is a cross-sectional view taken along line A-A of FIG. 3.

FIGS. 5A and 5B are cross-sectional views illustrating a usage example of a medical system according to the first embodiment, in which FIG. 5A is a cross-sectional view illustrating a state in which an inner wall of blood vessel is tented by a guiding catheter, and FIG. 5B is a cross-sectional view illustrating a state in which an assembly is inserted into a guiding catheter.

FIGS. 6A and 6B are cross-sectional views illustrating a usage example of a medical system according to the first embodiment, in which FIG. 6A illustrates a state in which a movement portion is made movable by a release portion, and FIG. 6B illustrates a state in which an inner wall of blood vessel is punctured by a puncture tool and a wire reaches a pericardial space.

FIGS. 7A and 7B are cross-sectional views illustrating a usage example of a medical system according to the first embodiment, in which FIG. 7A illustrates a state in which a wire is withdrawn after an inner wall of blood vessel is punctured by a puncture tool, and FIG. 7B illustrates a state in which the puncture tool is withdrawn after the inner wall of blood vessel is punctured by the puncture tool.

FIG. 8 is a flowchart illustrating an approach method by a medical system according to the first embodiment.

FIGS. 9A and 9B are cross-sectional views illustrating a usage example of a medical system according to a second embodiment, in which FIG. 9A illustrates a state in which an inner wall of blood vessel is tented by a guiding catheter, and FIG. 9B illustrates a state in which a movement portion is movable by a release portion.

FIG. 10 is a cross-sectional view illustrating a usage example of a medical system according to the second embodiment and illustrates a state in which an inner wall of blood vessel is punctured by a puncture tool and a wire reaches a pericardial space.

FIGS. 11A and 11B are cross-sectional views illustrating a modification example of a medical system according to the first embodiment, in which FIG. 11A illustrates a state in which an inner wall of blood vessel is tented by a guiding catheter, and FIG. 11B illustrates a state in which the inner wall of blood vessel is punctured by a puncture tool and a wire reaches a pericardial space.

### DETAILED DESCRIPTION

Set forth below with reference to the accompanying drawings is a detailed description of embodiments of a medical system and an approach method capable of approaching a pericardial space via a blood vessel. The dimensions of the drawings may be exaggerated and different from actual dimensions for convenience of description in some cases. In the present specification and the drawings, components having substantially the same functional configuration are denoted by the same reference numeral, and the redundant description will not be repeated. In the present specification, a side to be inserted into a lumen is referred to as a “distal side”, and a side of a hand operation portion is referred to as a “proximal side”.

### First Embodiment

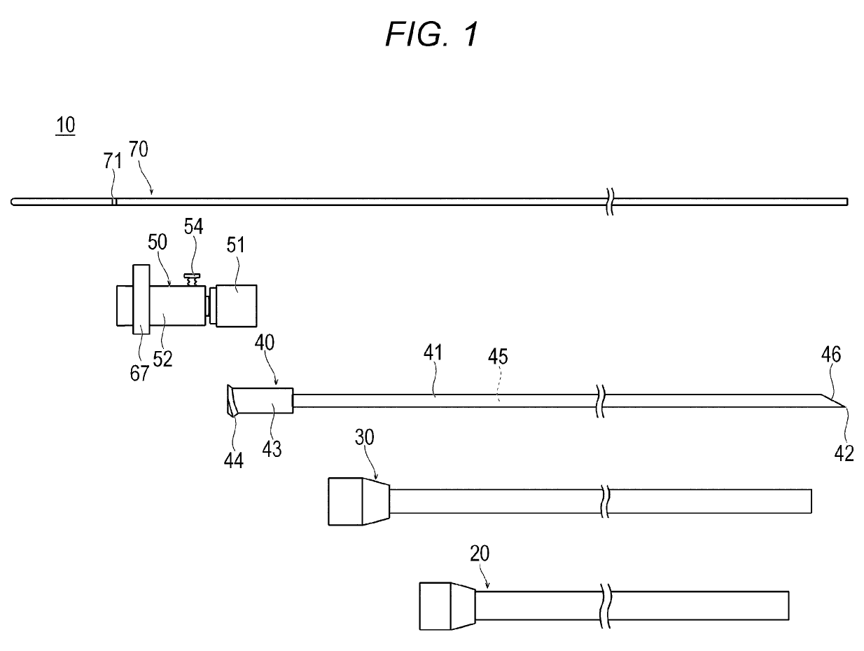

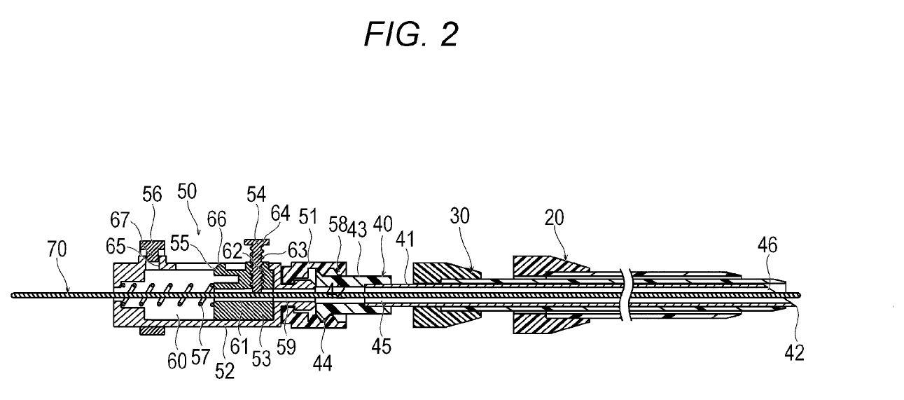

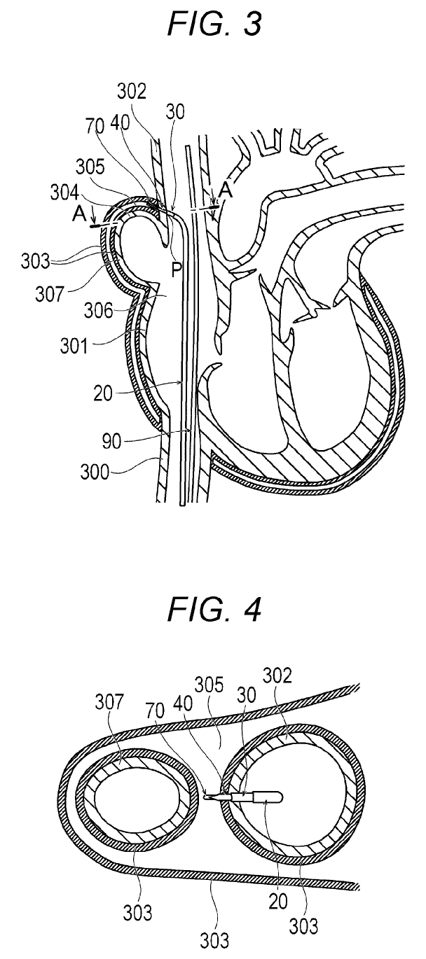

As illustrated in FIGS. 1 and 2, a medical system 10 according to a first embodiment of the present disclosure is a system for approaching a pericardial space 305 between two layers of the pericardium 303 constituting the pericardial sac covering the myocardium 304 via a blood vessel. The medical system 10 includes a backup catheter 20, a guiding catheter (catheter) 30, a puncture tool 40, a biasing tool 50, and a wire 70 (visualization means). As illustrated in FIG. 3, the medical system 10 is used together with an imaging device 90.

As illustrated in FIGS. 1 to 4, the backup catheter 20 is used to hold the guiding catheter 30 near the target position of a blood vessel or a heart to be punctured. The backup catheter 20 can be a known catheter but may be a dedicated catheter having a curved distal end.

The guiding catheter 30 is used to guide the puncture tool 40 near the target position of a blood vessel or a heart to be punctured. The guiding catheter 30 can be a known catheter but may be a dedicated catheter.

The imaging device 90 is a device that is percutaneously inserted into the inside of the blood vessel or the heart together with the backup catheter 20, the guiding catheter 30, the puncture tool 40, the wire 70, and the like, and acquires a tomographic image using intravascular ultrasound (IVUS). The imaging device 90 can be used to visually identify the position of the medical system 10 in the body. The imaging device 90 may be an X-ray imaging device capable of imaging a cross-section of a living body from outside the body by X-rays.

The puncture tool 40 is a tool that punctures the myocardium 304 from a heart chamber 306 to approach the pericardial space 305. The puncture tool 40 includes an elongated hollow shaft 41 having a puncture needle 42 at a distal end of the shaft 41, and a hub 43 to which a proximal portion of the shaft 41 is fixed. The puncture needle 42 can be formed, for example, by obliquely cutting the distal end of the shaft 41. The shape of the puncture needle 42 is not particularly limited as long as the living body can be punctured, and may be, for example, a conical shape, a knife-shaped flat plate, or a shovel-shaped curved surface plate. The cross-sectional shape of the puncture needle 42 may not be circular. The puncture needle 42 may be an electrode capable of emitting energy such as electric current or heat. In this case, the puncture needle 42 may not be sharp. The hub 43 has a male screw-shaped protrusion 44 on the outer peripheral surface of the hub 43. The shaft 41 and the hub 43 are formed with a lumen 45 penetrating from the distal portion to the proximal portion of the puncture tool 40. The puncture tool 40 has a distal opening 46 communicating with the lumen 45 at the distal portion of . The distal opening 46 is formed on an inclined distal surface of the distal portion of the puncture tool 40 on which the puncture needle 42 is formed.

The constituent material of the shaft 41 is preferably relatively rigid, and the constituent material of the shaft 41 is preferably, for example, metals such as stainless steel, tantalum, titanium, platinum, gold, and tungsten; polyolefins such as polyethylene and polypropylene; polyamide; polyester such as polyethylene terephthalate; fluorine-based polymers such as polytetrafluoroethylene (PTFE) and ethylene tetrafluoroethylene copolymer (ETFE); polyetheretherketone (PEEK), and polyimide.

The biasing tool 50 includes a connection portion 51 that can be connected to the hub 43, a housing 52 disposed on the proximal side of the connection portion 51, a movement portion 53 that can move in an axial direction with respect to the housing 52, a wire fixing portion 54 that can fix the wire 70 to the movement portion 53, a stopper 55 that releasably stops the movement portion 53 with respect to the housing 52 at a predetermined position in the axial direction, a release portion 56 that releases the stopper 55, and a biasing portion 57 that biases the movement portion 53 in a distal direction with respect to the housing 52.

The connection portion 51 has a tubular shape and is connected to the distal portion of the housing 52 so as to be rotatable in the circumferential direction with respect to the housing 52. The outer peripheral surface of the connection portion 51 is a portion that the operator rotationally operates to connect the connection portion 51 to the hub 43. The connection portion 51 has a female screw 58 on the inner peripheral surface of the connection portion 51 that can be screwed into the protrusion 44 on the outer peripheral surface of the hub 43.

The housing 52 is connected to the proximal portion of the connection portion 51. The housing 52 has a through hole 59 through which the wire 70 can pass. In a part of the through hole 59, a rail 60, on which the movement portion 53 is disposed to be movable in the axial direction, is formed.

The movement portion 53 is a portion to which the wire 70 is fixed and which moves together with the wire 70. The movement portion 53 is accommodated in the rail 60 of the housing 52 and is movable in the axial direction along the rail 60. The movement portion 53 has a fixing hole 61 penetrating in the axial direction and a fixing screw hole 62 which is a hole abutting the fixing hole 61 perpendicularly to the axial direction. The wire 70 can pass through the fixing hole 61. A fixing screw 63 formed at the wire fixing portion 54 can be screwed into the fixing screw hole 62. The fixing screw 63 can enter the fixing hole 61 from the side when the operator rotates a screw head 64. Therefore, the fixing screw 63 can fix the wire 70 to the movement portion 53 by pressing the wire 70 passing through the fixing hole 61 against the inner wall surface of the fixing hole 61.

The biasing portion 57 is disposed on the proximal side of the rail 60 of the through hole 59 of the housing 52 with respect to the movement portion 53. The biasing portion 57 biases the movement portion 53 in the distal direction with respect to the housing 52. The biasing portion 57 can be, for example, a coil spring, but may be an elastic body such as rubber.

The stopper 55 releasably stops, at a predetermined position, the movement portion 53 that has moved along the rail 60 in the proximal direction with respect to the housing 52 against the biasing force of the biasing portion 57. The stopper 55 can include, for example, a hook 66 that is formed to protrude from the movement portion 53, can be bent in a radial direction perpendicular to the axial direction, and can be caught by an engagement portion 65 of the housing 52. The engagement portion 65 is formed in a hole penetrating from the inner surface of the rail 60 of the housing 52 to the outer surface perpendicular to the axial direction. The hook 66 may be formed on the housing 52 side, and the engagement portion 65 may be formed on the movement portion 53 side. The configuration of the stopper 55 is not particularly limited as long as the movement portion 53 can be releasably stopped at a predetermined position with respect to the housing 52.

The release portion 56 is a portion that releases a state in which the movement portion 53 is stopped by the stopper 55 with respect to the housing 52. For example, the release portion 56 is connected to the housing 52 so as to be movable in a direction perpendicular to the axial direction without moving in the axial direction. For example, the release portion 56 is slidably disposed in a groove formed in a direction perpendicular to the axial direction on the outer peripheral surface of the housing 52. The release portion 56 has a protrusion portion 67 that can enter the engagement portion 65 from the outside in the radial direction. The protrusion portion 67 can push out the hook 66 engaged with the engagement portion 65 from the engagement portion 65 to release the engagement. The configuration of the release portion 56 is not particularly limited as long as a state in which the movement portion 53 is stopped with respect to the housing 52 can be released.

The wire 70 is an elongated and flexible member and is formed to have a length and an outer diameter capable of penetrating the lumen 45 of the puncture tool 40. The wire 70 has ultrasonic echogenicity and/or X-ray radiopacity so as to be imaged by the imaging device 90. The wire 70 may be a guidewire capable of guiding other tools.

The constituent material of the wire 70 is preferably flexible and relatively rigid, and the constituent material of the wire 70 is preferably, for example, metals such as stainless steel, tantalum, titanium, platinum, gold, and tungsten; a shape memory alloy, such as a Ni-Ti alloy, to which a shape memory effect or superelasticity is imparted; polyolefins such as polyethylene and polypropylene; polyamide; polyester such as polyethylene terephthalate; fluorine-based polymers such as polytetrafluoroethylene (PTFE) and ethylene tetrafluoroethylene copolymer (ETFE); polyetheretherketone (PEEK), and polyimide. The wire 70 may include an X-ray radiopaque material at least partially (for example, near the distal end of the wire 70). The X-ray radiopaque material is preferably formed of, for example, at least one metal or two or more alloys selected from the group consisting of gold, platinum, iridium, tungsten or alloys of gold, platinum, iridium, and tungsten, and a silver-palladium alloy.

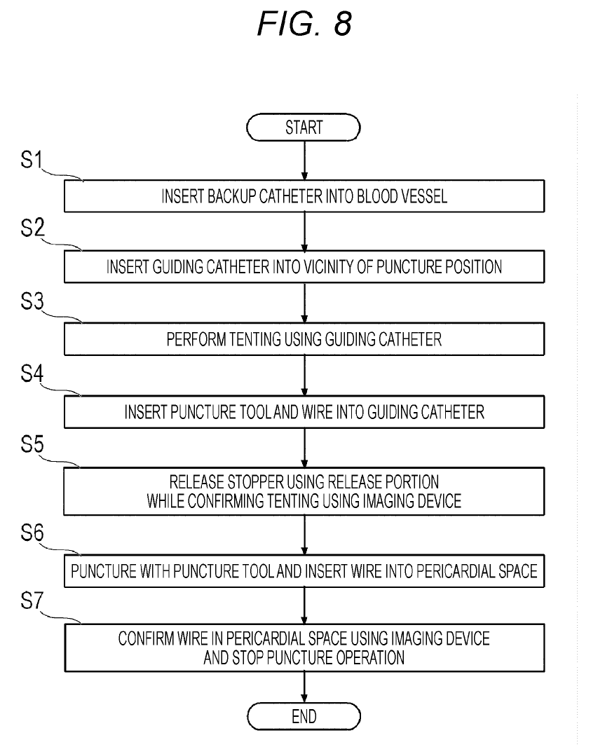

Next, a usage example of the medical system 10 according to the first embodiment will be described with reference to a flowchart illustrated in FIG. 8.

As illustrated in FIGS. 3, 4 and 5A,, the operator inserts the imaging device 90 and the backup catheter 20 percutaneously into the blood vessel (S1), and causes the distal end of the backup catheter 20 to reach the inside of the heart chamber 306 (for example, the right atrium 301) or the inside of the blood vessel (for example, the superior vena cava 302) through, for example, the inferior vena cava 300 while confirming the position of the backup catheter 20 using the imaging device 90. Next, the operator inserts the guiding catheter 30 into the lumen of the backup catheter 20 and causes the distal end of the guiding catheter 30 to protrude from the distal end of the backup catheter 20. The operator causes the distal end of the guiding catheter 30 to reach a predetermined position while confirming the position of the guiding catheter 30 using the imaging device 90 (S2). At this time, the operator places the distal end of the guiding catheter 30 at a puncture position P in the heart chamber 306 or the blood vessel at which the puncture tool 40 can approach the pericardial space 305 located between two layers of the pericardium 303 constituting the pericardial sac. The puncture position P is preferably a position where the pericardial space 305 to be the target of the approach is wide and the puncture tool 40 can easily approach. As an example, the target pericardial space 305 is preferably present between the superior vena cava 302 and the right atrial appendage 307. In this case, the puncture position P is located in the superior vena cava 302. The pericardial space 305 to be the approach target is not particularly limited. Therefore, the puncture position P may be located in the heart instead of the blood vessel.

Next, the operator presses the inner wall of the heart toward the pericardial space 305 with the distal end of the guiding catheter 30 at the puncture position P to perform tenting (S3). Thus, the inner wall of the heart enters a locally pressed state (tenting state) while generating a repulsive force.

Next, the operator assembles the puncture tool 40, the biasing tool 50, and the wire 70, and inserts the puncture tool 40 and the wire 70 into the lumen of the guiding catheter 30 (S4). In the assembled state, the female screw 58 of the connection portion 51 of the biasing tool 50 is engaged with the protrusion 44 of the hub 43 of the puncture tool 40, and thus the biasing tool 50 is connected to the puncture tool 40. The movement portion 53 of the puncture tool 40 moves in the proximal direction with respect to the housing 52, and the hook 66 of the stopper 55 is caught by the engagement portion 65 of the housing 52. In the housing 52, the biasing portion 57 is contracted in the axial direction on the proximal side with respect to the movement portion 53. The wire 70 passes through the lumen 45 of the puncture tool 40 and the through hole 59 and the fixing hole 61 of the biasing tool 50. The wire 70 is fixed to the movement portion 53 by the wire fixing portion 54 in which the fixing screw 63 is screwed into the fixing screw hole 62. The distal end of the wire 70 is disposed at a position substantially coinciding with the distal end of the puncture tool 40 or at a position slightly closer to the proximal side than the distal end of the puncture tool 40. An identification marker 71 (see FIG. 1) having a color different from its surroundings may be disposed on the surface of the wire 70 that matches a specific position (for example, proximal position) of the puncture tool 40 such that the operator can easily grasp the appropriate position of the wire 70.

Instead of inserting the puncture tool 40 and the wire 70 into the lumen of the guiding catheter 30 in a state in which the puncture tool 40, the biasing tool 50, and the wire 70 are combined, the operator may insert the guiding catheter 30 into the lumen of the backup catheter 20, insert the single puncture tool 40 into the lumen of the guiding catheter 30, and then insert the wire 70 into the lumen 45 of the puncture tool 40 in a state in which the biasing tool 50 and the wire 70 are combined. In this case, the wire 70 is preferably fixed at an appropriate position with respect to the biasing tool 50 such that the position of the distal end of the wire 70 with respect to the distal end of the puncture tool 40 is appropriate.

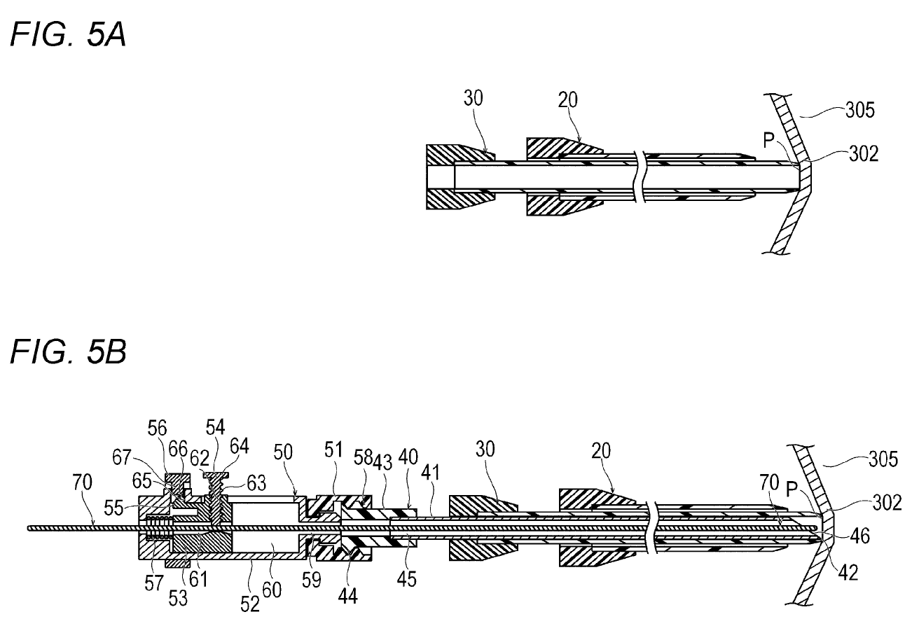

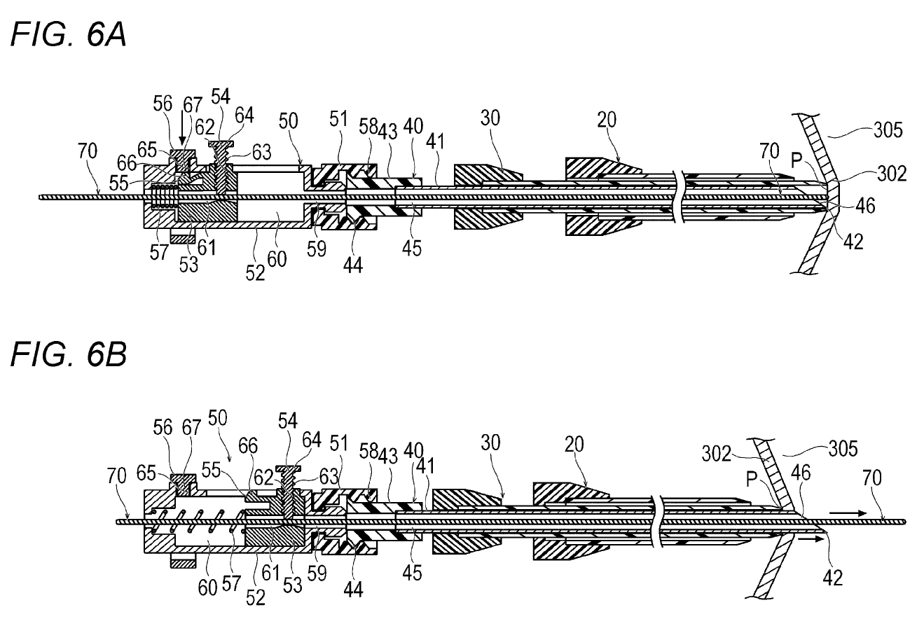

As illustrated in FIG. 5B, the operator disposes the distal ends of the puncture tool 40 and the wire 70 in the assembled state in the lumen of the guiding catheter 30 in the vicinity of the puncture position P. Next, the operator operates the release portion 56 to release the stopper 55 as illustrated in FIG. 6A while imaging the tented inner wall of the blood vessel or the tented inner wall of the heart using the imaging device 90 (S5). Thus, the hook 66 is disengaged from the engagement portion 65, the movement portion 53 is biased in the distal direction by the biasing portion 57, and the puncture preparation state is established. Therefore, the wire 70 fixed to the movement portion 53 by the fixing portion is biased in a direction to be pushed out from the distal end of the puncture needle 42 and abuts the inner wall of the blood vessel or the inner wall of the heart.

In a state in which the inner wall of the blood vessel or the inner wall of the heart is tented by the guiding catheter 30 and in a state in which the wire 70 is biased by the biasing tool 50, the operator operates the puncture tool 40 to move the puncture tool 40 in the distal direction, and punctures the inner wall of the blood vessel or the inner wall of the heart with the puncture needle 42 (S6). As illustrated in FIG. 6B, immediately after the puncture needle 42 reaches the inside of the pericardial space 305 together with the distal opening 46, the wire 70 biased by the biasing tool 50 protrudes outward (in the distal direction) from the lumen 45 of the puncture tool 40 through the distal opening 46 and reaches the inside of the pericardial space 305. When the wire 70 reaches the inside of the pericardial space 305, the movement portion 53 moves in the distal direction due to the biasing force of the biasing portion 57 inside the housing 52 of the biasing tool 50. The operator can rather easily confirm the arrival of the wire 70 into the pericardial space 305 in real time using the image captured by the imaging device 90 and can stop the movement of the puncture tool 40 in the distal direction (S7). That is, the operator can immediately recognize that the distal opening 46 of the puncture tool 40 has reached the inside of the pericardial space 305 and stop the advancement of the puncture needle 42 (puncture operation). Thus, it is possible to help prevent the puncture needle 42 from being excessively advanced into the outer layer of the pericardium 303 or to help prevent the distal opening 46 from being buried in the adjacent myocardium (right atrial appendage 307), and it is possible to approach the pericardial space 305 relatively safely and easily. In a case where the wire 70 has X-ray radiopacity, the operator may confirm the arrival of the wire 70 into the pericardial space 305 using the image captured by the X-ray imaging device. The advancement of the wire 70 fixed to the movement portion 53 by the wire fixing portion 54 automatically stops after reaching the inside of the pericardial space 305 by the movement portion 53 abutting the distal portion of the housing 52. Therefore, it is possible to prevent the wire 70 from penetrating the outer layer of the pericardium 303.

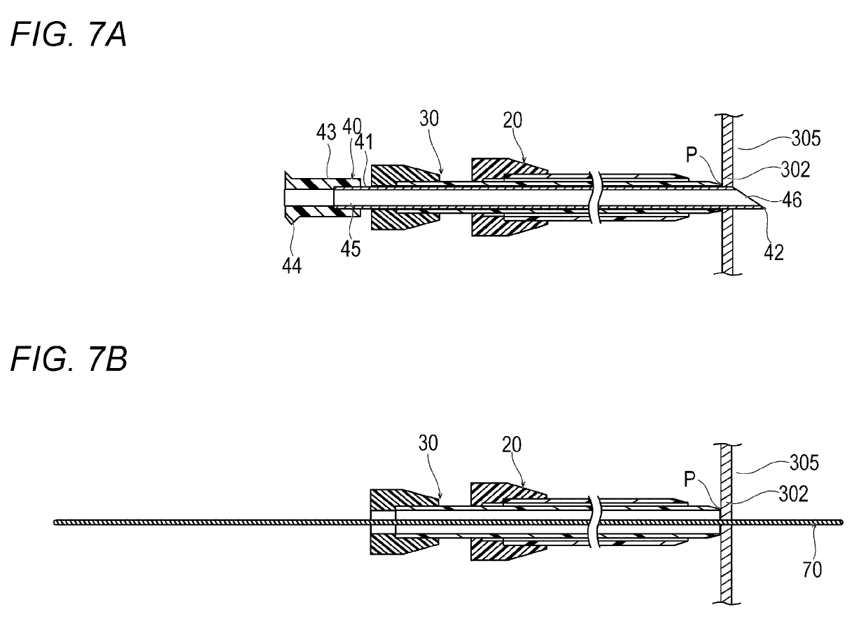

Next, as illustrated in FIG. 7A, the operator withdraws the wire 70 together with the biasing tool 50 in a state in which the puncture tool 40 remains in the pericardial space 305. Thus, it is possible to administer agents into the pericardial space 305 through the lumen 45 of the puncture tool 40, insert a therapeutic tool , and drain, for example, a body fluid. As illustrated in FIG. 7B, the operator may use the wire 70 as a guidewire by withdrawing the puncture tool 40 and the wire 70 without withdrawing the wire 70. For example, the operator can insert the therapeutic tool into the pericardial space 305 along the wire 70.

After puncturing the inner wall of the blood vessel or the inner wall of the heart with the puncture needle 42 at the puncture position P, the operator may cause the distal end of the guiding catheter 30 to pass through the hole formed by puncture and reach the pericardial space 305. In this case, after withdrawing the puncture tool 40, the operator can administer agents into the pericardial space 305, insert the therapeutic tool, or drain the body fluid through the lumen of the guiding catheter 30.

As described above, the medical system 10 according to the first embodiment includes: an elongated guiding catheter 30 having flexibility; an elongated puncture tool 40 that is insertable into the guiding catheter 30, has a puncture needle 42 at an distal portion, and includes a lumen 45 extending from a proximal portion to the distal portion and a distal opening 46 communicating with the lumen 45 at the distal portion; a biasing tool 50 that includes a movement portion 53 movable for applying a biasing force toward a distal direction to a material passing through the lumen 45 and a biasing portion 57 that biases the movement portion 53; and a releasable stopper 55 that is capable of fixing the movement portion 53 at a predetermined position. Thus, the medical system 10 can push out the material passing through the lumen 45 of the puncture tool 40 from the distal opening 46 by releasing the stopper 55 and applying the biasing force to the material using the movement portion 53. Therefore, since the medical system 10 can cause the material passing through the lumen 45 of the puncture tool 40 to reach the pericardial space 305 by puncturing the inner wall of the heart or the inner wall of the blood vessel with the puncture tool 40 from the inside of the heart chamber 306 or from the inside of the blood vessel, the puncture tool 40 or the wire 70 can be safely and easily caused to approach the pericardial space 305 while observing the material reaching the pericardial space 305 through the lumen 45.

The medical system 10 further includes a visualization means having ultrasonic echogenicity or X-ray radiopacity as the material passing through the lumen 45. Thus, in the medical system 10, the visualization means that reaches the pericardial space 305 through the lumen 45 can be observed by using the imaging device 90 using ultrasonic waves or X-rays.

The visualization means, can be, for example, the wire 70. Thus, in the medical system 10, the wire 70 reaching the pericardial space 305 through the lumen 45 can be observed using the imaging device 90.

The biasing tool 50 is detachable from the puncture tool 40. Thus, since the biasing tool 50 can be attached to the puncture tool 40 when necessary and removed from the puncture tool 40 when unnecessary, the workability of the puncture tool 40 can be improved.

A biasing tool 50can include a wire fixing portion 54 capable of fixing the wire 70 to the movement portion 53. Thus, the medical system 10 can relatively easily fix the wire 70 to the movement portion 53 and move the wire together with the movement portion 53.

The approach method according to the present embodiment is a method for approaching a pericardial space 305 from an inside of a heart chamber 306 or an inside of a blood vessel, the method including: preparing a flexible elongated guiding catheter 30 and an elongated puncture tool 40 which is insertable into the guiding catheter 30, has a puncture needle 42 at a distal end, and includes a lumen 45 extending from a proximal portion to a distal portion and a distal opening 46 communicating with the lumen 45 at the distal portion; inserting the guiding catheter 30 into the blood vessel; delivering the guiding catheter 30 to a puncture position P in the heart chamber 306 or the blood vessel from which the puncture tool 40 inserted in the guiding catheter 30 is capable of performing a puncture toward the pericardial space 305 (S2); at the puncture position P, pressing an inner wall of a heart or an inner wall of the blood vessel toward the pericardial space 305 at the distal portion disposed at the distal end of the guiding catheter 30 to make a tenting state (S3); making a puncture preparation state by imaging the pressed inner wall of the heart or the pressed inner wall of the blood vessel using an imaging device 90, biasing a visualization means, which has ultrasonic echogenicity or X-ray radiopacity and is inserted in the lumen 45, in a direction to push the visualization means out of the lumen 45 from the distal opening 46 of the puncture tool 40, and restricting movement of the visualization means to the outside from the distal opening 46 (S5); and in the tenting state and the puncture preparation state, puncturing the inner wall of the heart or the inner wall of the blood vessel with the puncture needle 42 through the lumen of the guiding catheter 30 to release the restriction on the movement of the visualization means, and inserting the biased visualization means into the pericardial space 305 through the distal opening 46 of the puncture tool 40 while imaging by the imaging device 90 (S6). Thus, in the present approach method, by puncturing the inner wall of the heart or the inner wall of the blood vessel from the heart chamber 306 with the puncture tool 40, the visualization means passing through the lumen 45 of the puncture tool 40 can reach the pericardial space 305. Therefore, in the present approach method, the puncture tool 40 and the visualization means can approach the pericardial space 305 relatively safely and easily by observing the visualization means using the imaging device 90.

### Second Embodiment

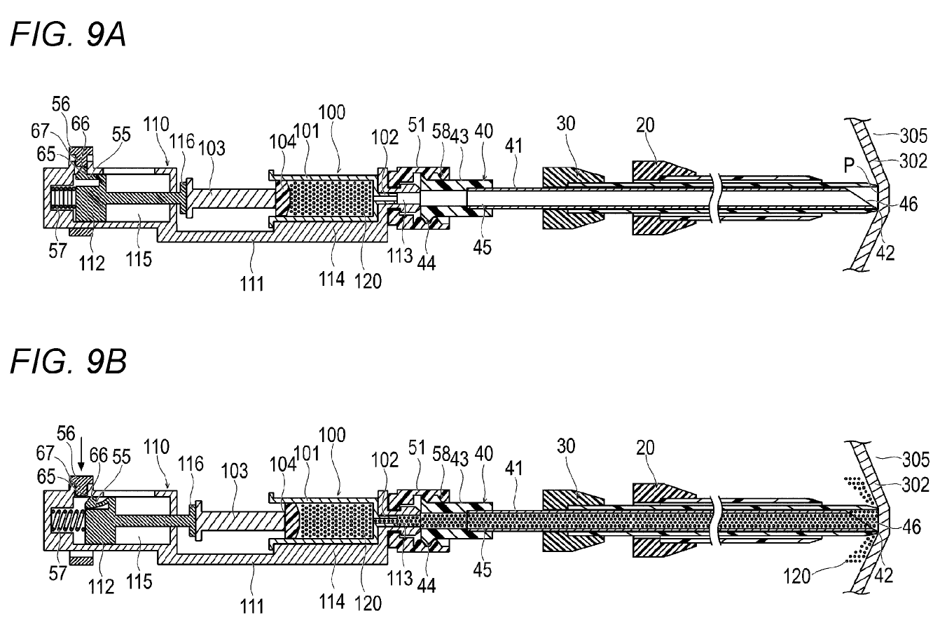

As illustrated in FIG. 9A, a medical system 10 according to a second embodiment is different from that of the first embodiment in that a microbubble 120 is used as a visualization means instead of the wire 70. The backup catheter 20, the guiding catheter 30, and the puncture tool 40 in the second embodiment are the same as those in the first embodiment.

The microbubble 120 as the visualization means is provided by a syringe 100 in a state of being mixed with a liquid. The syringe 100 includes an outer cylinder 101 that stores a liquid containing the microbubbles 120, a nozzle 102 located on the distal side with respect to the outer cylinder 101 to discharge the liquid containing the microbubbles 120, a plunger 103 inserted into the outer cylinder 101 from the proximal side to push out the liquid containing the microbubbles 120, and a gasket 104 fixed to the distal end of the plunger 103.

The microbubbles are minute bubbles each having a diameter of 10 μm to several tens of μm (i.e., up to 90 µm). The gas forming the microbubbles 120 can be, for example, air, carbon dioxide gas, or the like. Since the microbubbles consist of gas, the microbubbles have ultrasonic echogenicity. In a case where the gas is carbon dioxide gas, the microbubbles also have X-ray radiopacity. The liquid with which the microbubbles are mixed can be, for example, saline (saline solution), or a cytoprotective solution such as dimethyl sulfoxide (DMSO) or glycerol.

A biasing tool 110 includes a connection portion 51 that can be connected to a hub 43, a housing 111 disposed on the proximal side of the connection portion 51, a movement portion 112 that can move in an axial direction with respect to the housing 111, a stopper 55 that releasably stops the movement portion 112 with respect to the housing 111 at a predetermined position in the axial direction, a release portion 56 that releases the stopper 55, and a biasing portion 57 that biases the movement portion 112 in a distal direction with respect to the housing 111.

The housing 111 is connected to the proximal portion of the connection portion 51. The housing 111 includes a connection hole 113 into which the nozzle 102 of the syringe 100 can be inserted and which can communicate with the lumen 45 of the puncture tool 40, an outer cylinder holding portion 114 that holds the outer cylinder 101 of the syringe 100, and a rail 115 that holds the movement portion 112 movably in the axial direction.

The movement portion 112 is accommodated in the rail 115 of the housing 111 and is movable in the axial direction along the rail 115. The movement portion 112 has a pressing portion 116 that can press the plunger 103 of the syringe 100.

Next, a usage example of the medical system 10 according to the second embodiment will be described.

As in the first embodiment, the operator inserts the imaging device 90 and the backup catheter 20 percutaneously into the blood vessel, inserts the guiding catheter 30 into the lumen of the backup catheter 20, and causes the distal end of the guiding catheter 30 to protrude from the distal end of the backup catheter 20 to reach a predetermined position. At this time, the operator disposes the distal end of the guiding catheter 30 at the puncture position P in the heart chamber 306 or the blood vessel from which the puncture tool 40 can approach the pericardial space 305. Next, at the puncture position P, the operator presses the inner wall of the heart or the inner wall of the blood vessel toward the pericardial space 305 using the distal portion of the guiding catheter 30 to perform a tenting.

Next, the operator inserts into the lumen of the guiding catheter 30, the puncture tool 40 in a state in which the puncture tool 40, the biasing tool 110, and the syringe 100 are combined. In a state in which the puncture tool 40, the biasing tool 110, and the syringe 100 are combined, the female screw 58 of the connection portion 51 of the biasing tool 110 is inserted into the protrusion 44 of the hub 43 of the puncture tool 40, and the biasing tool 110 is connected to the puncture tool 40. The movement portion 112 of the biasing tool 110 moves in the proximal direction with respect to the housing 111, and the hook 66 of the stopper 55 is caught by the engagement portion 65 of the housing 111. In the housing 111, the biasing portion 57 is contracted in the axial direction on the proximal side with respect to the movement portion 112. The pressing portion 116 of the movement portion 112 is located on the proximal side of the plunger 103 of the syringe 100.

Instead of inserting the puncture tool 40 into the lumen of the guiding catheter 30 in a state in which the puncture tool 40, the biasing tool 110, and the syringe 100 are combined, the operator may insert the single puncture tool 40 into the lumen of the guiding catheter 30, and then insert the wire 70 into the lumen 45 of the puncture tool 40 in a state in which the biasing tool 110 and the wire 70 are combined.

Next, the operator operates the release portion 56 to release the stopper 55 as illustrated in FIG. 9B while imaging the tented inner wall of the blood vessel or the tented inner wall of the heart using the imaging device 90. Thus, the hook 66 is disengaged from the engagement portion 65, and the movement portion 112 is biased in the distal direction by the biasing portion 57. Therefore, the plunger 103 of the syringe 100 pressed by the pressing portion 116 of the movement portion 112 gradually moves in the distal direction. As a result, the liquid containing the microbubbles 120 is gradually discharged from the nozzle 102 of the syringe 100 and pushed out from the distal end of the puncture needle 42 of the puncture tool 40. The liquid containing the microbubbles 120 is discharged at a substantially constant flow rate in the blood vessel or in the heart chamber 306.

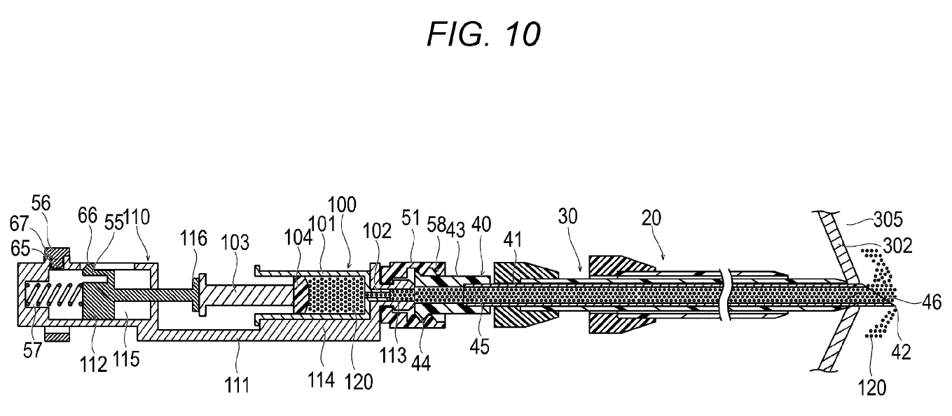

In a state in which the inner wall of the heart is tented by the guiding catheter 30 and the liquid containing the microbubbles 120 is biased by the biasing tool 110, the operator operates the puncture tool 40 to move the puncture tool 40 in the distal direction while performing imaging using the imaging device 90 and punctures the inner wall of the heart with the puncture needle 42. As soon as the puncture needle 42 reaches the inside of the pericardial space 305 together with the distal opening 46, the liquid containing the microbubbles 120 that has been biased by the biasing tool 110 and discharged in the blood vessel or the heart chamber 306 is discharged in the pericardial space 305 as illustrated in FIG. 10. The operator can rather easily confirm the discharge of the liquid containing the microbubbles 120 in the pericardial space 305 in real time using the image captured by the imaging device 90 and can stop the movement of the puncture tool 40 in the distal direction. That is, the operator can immediately recognize that the distal opening 46 of the puncture tool 40 has reached the inside of the pericardial space 305 and stop the advancement of the puncture needle 42 (puncture operation). Thus, it is possible to prevent the puncture needle 42 from being excessively advanced to penetrate the outer layer of the pericardium 303 or to prevent the distal opening 46 from being buried in the adjacent myocardium (right atrial appendage 307), and it is possible to approach the pericardial space 305 safely and easily.

Next, the operator withdraws the biasing tool 110 and the syringe 100 in a state in which the puncture tool 40 remains in the pericardial space 305. Thus, it is possible to administer agents into the pericardial space 305 through the lumen 45 of the puncture tool 40, insert a therapeutic tool or the like, and drain a body fluid.

As described above, in the medical system 10 according to the second embodiment, the visualization means may be a liquid containing a plurality of microbubbles 120. Thus, in the medical system 10, the microbubbles 120 reaching the pericardial space 305 through the lumen 45 can be observed using the imaging device 90.

The present disclosure is not limited to only the embodiment described above, and those skilled in the art can make various modifications within the technical idea of the present invention. For example, the backup catheter 20 may not be included in the medical system 10.

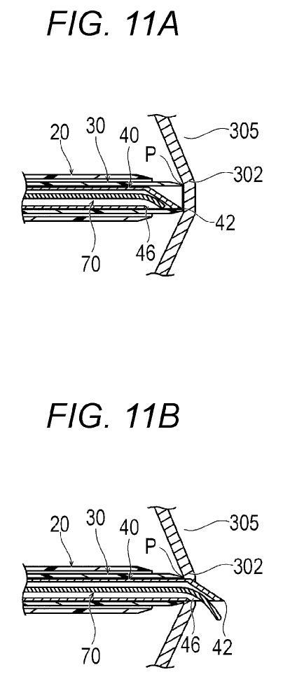

The distal opening 46 of the puncture tool 40 may be formed at a position different from the inclined distal surface of the distal portion of the puncture tool 40. For example, as illustrated in FIG. 11A, the distal opening 46 may be formed as a side hole penetrating from the outer wall surface to the inner wall surface slightly on the proximal side with respect to the most distal end of the puncture tool 40. In this case, the distal end of the biased wire 70 is guided by the distal inner wall surface of the puncture needle 42 to protrude laterally from the distal opening 46 and abuts the inner peripheral surface of the guiding catheter 30. Therefore, the movement is restricted so as not to protrude any more. It is preferable that the wire 70 is not biased by the biasing tool 50 such that a load is not applied to the wire 70 before the tenting state.

When the distal opening 46 of the puncture tool 40 is located on the distal side with respect to the distal end of the guiding catheter 30, as illustrated in FIG. 11B, the wire 70 biased by the biasing tool 50 completely protrudes to the outside from the distal opening 46 which is a side hole, and reaches the inside of the pericardial space 305.

The detailed description above describes embodiments of a medical system and an approach method capable of approaching a pericardial space via a blood vessel. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A medical system comprising:
an elongated catheter having flexibility;
an elongated puncture tool that is insertable into the catheter, the puncture tool including a puncture needle at a distal portion of the puncture tool, and wherein the puncture tool includes a lumen extending from a proximal portion of the puncture tool to the distal portion of the puncture tool and a distal opening communicating with the lumen at the distal portion of the puncture tool;
a biasing tool that includes a movement portion movable for applying a biasing force toward a distal direction to a material passing through the lumen and a biasing portion that biases the movement portion; and
a releasable stopper that is configured to fix the movement portion of the biasing portion at a predetermined position.

2. The medical system according to claim 1 further comprising:
a visualization means having ultrasonic echogenicity or X-ray radiopacity as the material passing through the lumen.

3. The medical system according to claim 2, wherein the visualization means is a wire.

4. The medical system according to claim 2, wherein the visualization means is a liquid containing a plurality of microbubbles.

5. The medical system according to claim 1, wherein the biasing tool is detachable from the puncture tool.

6. The medical system according to claim 3, wherein the biasing tool includes a wire fixing portion configured to fix the wire to the movement portion.

7. The medical system according to claim 1, wherein the biasing tool includes a connection portion configured to be connected to a hub to which a proximal portion of the puncture tool is fixed, a housing disposed on the proximal side of the connection portion, the movement portion configured to move in the axial direction with respect to the housing, a wire fixing portion configured to fix a wire to the movement portion, the releasable stopper configured to releasably stop the movement portion with respect to the housing at the predetermined position in the axial direction, a release portion configured to release the releasable stopper, and a biasing portion configured to bias the movement portion in a distal direction with respect to the housing.

8. The medical system according to claim 7, wherein the connection portion has a tubular shape and is connected to the distal portion of the housing and is configured to rotated in a circumferential direction with respect to the housing.

9. The medical system according to claim 8, wherein an outer peripheral surface of the connection portion is a portion that the operator rotationally operates to connect the connection portion to the hub.

10. The medical system according to claim 9, wherein the connection portion includes a female screw on an inner peripheral surface of the connection portion configured to be screwed into a protrusion on the outer peripheral surface of the hub.

11. The medical system according to claim 7, wherein the housing is connected to the proximal portion of the connection portion, the housing includes a through hole through which the wire is configured to pass, and in a part of the through hole, a rail on which the movement portion is disposed to be movable in the axial direction, is formed.

12. The medical system according to claim 11, wherein the movement portion is a portion to which the wire is fixed and which moves together with the wire and is accommodated in the rail of the housing and is configured to be movable in the axial direction along the rail, and wherein the movement portion has a fixing hole penetrating in the axial direction and a fixing screw hole abutting the fixing hole perpendicularly to the axial direction.

13. The medical system according to claim 11, wherein the biasing portion is disposed on the proximal side of the rail of the through hole of the housing with respect to the movement portion, the biasing portion configured to bias the movement portion in the distal direction with respect to the housing.

14. A medical system comprising:
an elongated catheter;
an elongated puncture tool configured to be inserted into a lumen of the catheter, the puncture tool including an elongated shaft having a puncture needle at a distal end of the elongated shaft, and wherein the elongated shaft includes a lumen extending from a proximal end of the elongated shaft to the distal end of the elongated shaft and a distal opening communicating with the lumen at the distal end of the elongated shaft; and
a biasing tool that includes a movement portion configured to be movable for applying a biasing force toward a distal direction to a material passing through the lumen and a biasing portion configured to bias the movement portion.

15. The medical system according to claim 14, further comprising:
a releasable stopper configured to fix the movement portion of the biasing tool at a predetermined position.

16. The medical system according to claim 14, further comprising:
a wire, the wire having ultrasonic echogenicity or X-ray radiopacity as the material passes through the lumen.

17. The medical system according to claim 16, wherein the biasing tool includes a wire fixing portion configured to fix the wire to the movement portion.

18. The medical system according to claim 15, wherein the biasing tool includes a connection portion configured to be connected to a hub to which a proximal portion of the puncture tool is fixed, a housing disposed on the proximal side of the connection portion, the movement portion configured to move in the axial direction with respect to the housing, a wire fixing portion configured to fix a wire to the movement portion, the releasable stopper configured to releasably stop the movement portion with respect to the housing at the predetermined position in the axial direction, a release portion configured to release the releasable stopper, and a biasing portion configured to bias the movement portion in a distal direction with respect to the housing.

19. The medical system according to claim 18, wherein the connection portion has a tubular shape and is connected to the distal portion of the housing and is configured to rotated in a circumferential direction with respect to the housing, and an outer peripheral surface of the connection portion is a portion that the operator rotationally operates to connect the connection portion to the hub.

20. An approach method for approaching a pericardial space from an inside of a heart chamber or an inside of a blood vessel, the method comprising:
preparing an elongated catheter having flexibility and an elongated puncture tool which is insertable into the catheter, the puncture tool having a puncture needle at a distal end of the puncture tool, and includes a lumen extending from a proximal portion of the puncture tool to a distal portion of the puncture tool and a distal opening communicating with the lumen at the distal portion of the puncture tool;
inserting the catheter into the blood vessel and delivering the catheter to a puncture position in the heart chamber or the blood vessel from which the puncture tool inserted into the catheter performing a puncture toward the pericardial space;
at the puncture position, pressing an inner wall of a heart or an inner wall of the blood vessel toward the pericardial space at the distal portion disposed at the distal end of the catheter to make a tenting state;
making a puncture preparation state by imaging the pressed inner wall of the heart or the pressed inner wall of the blood vessel using an imaging device, biasing a visualization means, which has ultrasonic echogenicity or X-ray radiopacity and is inserted into the lumen, in a direction to push the visualization means out of the lumen from the distal opening of the puncture tool, and restricting movement of the visualization means to an outside from the distal opening; and
in the tenting state and the puncture preparation state, puncturing the inner wall of the heart or the inner wall of the blood vessel with the puncture needle through the lumen of the catheter to release the restriction on the movement of the visualization means, and inserting the biased visualization means into the pericardial space through the distal opening of the puncture tool while imaging by the imaging device.
